(19) United States
(12) Patent Application Publication
(10) Pub. No.: US 2002/0168293 A1
(43) Pub. Date: Nov. 14, 2002

(54) Chemical protective materials testing system

(75) Inventor: Dean Smith, Dover-Foxcroft, ME (US)

Correspondence Address: PATRICK R. SCANLON, PIERCE ATWOOD, ONE MONUMENT SQUARE, PORTLAND, ME, 04101, US

(73) Assignee: Sensor Research and Development Corporation

(21) Appl. No.: 10/104,425

(22) Filed: Mar. 22, 2002

Related U.S. Application Data
(60) Provisional application No. 60/278,073, filed on Mar. 22, 2001.

(51) Int. Cl. G01N 33/00

(52) U.S. Cl. 422/68.1; 422/99; 422/100; 422/942

(57) ABSTRACT

A system for testing chemically resistant materials includes a sample holding assembly for holding a sample of a material to be tested. The sample holding assembly defines a plurality of test chambers wherein each test chamber has a portion of the sample disposed therein. An analyte injection unit is included for dispensing a controlled amount of an analyte into each test chamber. The system further includes a plurality of analyte detection units. Each one of the detection units is in fluid communication with a corresponding one of the test chambers for sensing analyte as it permeates the material sample.

### CROSS REFERENCES TO RELATED APPLICATIONS

This application claims the benefit of U.S. Provisional Application No. 60/278,073, filed Mar. 22, 2001.

### BACKGROUND OF THE INVENTION

This invention relates generally to materials testing and more particularly to systems for testing chemically resistant materials used in personal protective equipment.

Workers involved in the production, use, and transportation of chemicals can be exposed to numerous compounds capable of causing harm upon contact with the human body. The deleterious effects of these chemicals can range from immediate death, to acute trauma such as skin irritation and burn, to chronic degenerative disease, such as cancer or pulmonary fibrosis. Since engineering controls may not eliminate all possible exposures, attention is often placed on reducing the potential for direct contact through the use of personal protective equipment (e.g., chemical suits, protective gloves and the like) that resists chemical permeation, penetration, and degradation. Personal protective equipment is also used by military personnel as a safeguard against chemical and biological warfare.

To determine levels of protection afforded by personal protective equipment, effective certification testing must be performed. Such testing involves evaluation of the chemical resistance, in terms of breakthrough, permeation, and degradation, of materials used for personal protective equipment. Current standards governing protective material testing of permeable samples include TOP 8-2-501, ASTM F739, ASTM F903, etc.

Current methods for evaluating chemically resistant materials are generally slow, burdensome costly and inconsistent. Although the testing methods are standardized there is no standard testing system available. The result is inter-laboratory inconsistency and unreliable material performance data.

The most common system currently available for testing chemically resistant materials uses an impinger system. Multiple swatches of the material to be tested are placed into test chambers. A few drops of a chemical agent are manually placed on the external (away from skin) surface of each swatch and the test chambers are closed to create a seal. Clean, conditioned air is passed through (through-flow) or across (non-through-flow) the external surface of the swatches. The chemical agent eventually permeates the swatches, where it is collected by mixing with a suitable solvent in an impinger. A sample of the solvent is collected periodically and permeation of the chemical agent on the internal (towards skin) surface of the swatches can be calculated from the concentration of the agent in the solvent using gas chromatography or similar methods.

Such systems have a number of drawbacks. For instance, the chemical agent is manually applied to the swatches under a separate laboratory hood with a lack of environmental control, which leads to significant temperature and humidity variations. This is a time-consuming procedure that results in inconsistent agent application and requires the technicians to work directly in hazard zones. The test chambers and impingers are also manually connected. The use of impingers requires human intervention for sample collection, tracking, analysis and reporting and does not provide real-time performance data. Instead, these systems provide discrete permeation data points (e.g., every 8 hours). This arrangement also leads to interrupted flow during testing.

Accordingly, there is a need for a system for testing chemically resistant materials that can safely and efficiently provide quality, real-time data and insight to material performance against chemical permeation. It is also desirable to have a testing system that provides a faster, more automated and economical method to accurately evaluate chemically resistant materials.

### SUMMARY OF THE INVENTION

The above-mentioned need is met by the present invention, which provides an integrated system for controlled, precision testing of chemically resistant materials subject to liquid and/or vapor analytes. The purpose of the system is to improve the quality, efficiency, accuracy, and reproducibility of the material permeation data over existing methods. The system provides a safer, non-interruptible, controlled environment to an array of test material. All system components (test material array, programmable analyte autoinjection, environmental control, and sensors) are contained inside a safe environmental enclosure allowing completely hands-free operation after sample loading. System parameters (relative humidity, temperature, pressure and flow) presented to the material under test are controlled to be constants. This allows the application of analyte onto the material to be the sole independent variable presented to permeation sensing mechanisms downstream. This system can provide valuable material permeation kinetics information (when does breakthrough occur?, what are the breakthrough mechanisms?, what are the instantaneous permeation rates?) that can be used by current protective clothing consumers to better gauge the safety performance of their ensembles and by manufacturers to tailor material designs for optimal chemical protection.

The present invention and its advantages over the prior art will become apparent upon reading the following detailed description and the appended claims with reference to the accompanying drawings.

### DESCRIPTION OF THE DRAWINGS

The subject matter that is regarded as the invention is particularly pointed out and distinctly claimed in the concluding part of the specification. The invention, however, may be best understood by reference to the following description taken in conjunction with the accompanying drawing figures in which:

FIG. 1 is a perspective view of a system for testing chemically resistant materials.

FIG. 2 is a perspective view of the analyte injection unit from the system of FIG. 1.

FIG. 3 is a cross-sectional view of the sample holding assembly from the system of FIG. 1.

FIG. 4 is a perspective view of the lower swatch array block of the sample holding assembly of FIG. 3.

FIG. 5 is a perspective view of a detection unit from the system of FIG. 1.

FIG. 6 is a perspective view of the upper swatch array block of an alternative embodiment of a system for testing chemically resistant materials.

FIG. 7 is a perspective view of the lower swatch array block of an alternative embodiment of a system for testing chemically resistant materials.

FIG. 8 is a perspective view of the upper swatch array block of another alternative embodiment of a system for testing chemically resistant materials.

FIG. 9 is a perspective view of the lower swatch array block of another alternative embodiment of a system for testing chemically resistant materials.

### DETAILED DESCRIPTION OF THE INVENTION

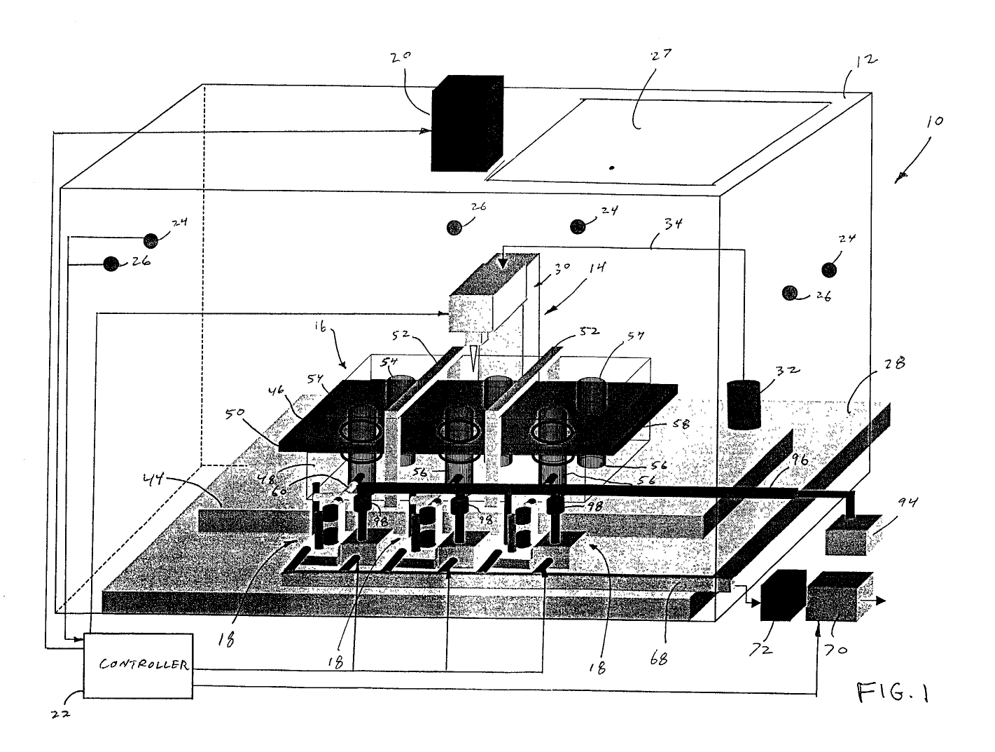

Referring to the drawings wherein identical reference numerals denote the same elements throughout the various views, FIG. 1 shows a system 10 for testing chemically resistant materials. The system 10 includes an environmental enclosure 12 that houses an analyte injection unit 14, a sample holding assembly 16 and an array of analyte detection units 18. The enclosure 12 is a gas tight structure sized to fit inside a laboratory fume hood so that exhaust from the system 10 can directly and safely be sent to the exhaust manifold of the fume hood. An input manifold 20 is included for introducing conditioned lab air into the enclosure 12. As is known in the art, the input manifold 20 includes filters, heaters, temperature sensors and humidity sensors that are controlled by a system controller 22 to provide clean, conditioned air at a constant temperature and humidity. Temperature sensors 24 and humidity sensors 26 are disposed inside the enclosure 12 and provide feedback to the controller 22 to maintain a precise testing environment. The enclosure 12 is provided with a door 27 to permit a sample or swatch of the material to be tested to be placed inside the enclosure 12. The enclosure 12 can also accommodate built-in, box style gloves (not shown) for safe manual manipulation of system components. To this end, the walls of the enclosure are made of a clear, see-through material.

The analyte injection unit 14 includes a base plate 28 located on the bottom of the enclosure 12 and a robotic autoinjector 30 extending above the base plate 28. The autoinjector 30 is programmable for X, Y, Z positional control (i.e., motion along three mutually orthogonal axes) under the control of the controller 22. An analyte source 32 for delivering analyte to the autoinjector 30 via conduit 34 is located externally of the enclosure 12. As used herein, “analyte” refers to a substance that is applied to a sample material to test the material's resistance to breakthrough, permeation, and degradation for the substance. Analytes can include chemical agents, toxic industrial chemicals and the like, and can be either liquid or vapor.

Referring to FIG. 2, the autoinjector 30 includes a vertical column 36 that can move laterally along the back edge of the base plate 28. A support arm 38 is mounted to the column 36 so as to extend over the base plate 28. The support arm 38 is capable of moving vertically with respect to the column 36. A syringe holder 40 is slidingly mounted for movement back-and-forth along the support arm 38. The syringe holder 40 supports a syringe 42 that receives analyte from the conduit 34 and can deposit a controlled amount of the analyte onto the test sample. The syringe 42 can provide a programmable injection amount; for example, a 1 microliter drop with a less than 5% volume standard deviation. Although a single syringe is shown in FIGS. 1 and 2, it should be noted that the syringe holder 40 could carry an array of syringes for simultaneous dispensing multiple analyte drops in a desired pattern.

Referring again to FIG. 1, the sample holding assembly 16 is mounted on the base plate 28, under the support arm 38. The sample holding assembly 16 includes a support plate 44 mounted directly on top of the base plate 28 and upper and lower swatch array blocks 46 and 48. The upper and lower swatch array blocks 46 and 48 are stacked on top of the support plate 44 with a sample 50 of the material to be tested sandwiched therebetween. Two clamping mechanisms 52 are attached to the support plate 44 and enclose the swatch array blocks 46 and 48 so as to press the upper swatch array block 46 down on the lower swatch array block 48 and securely clamp the material sample 50 in position. The sample holding assembly 16 is configured so as not to interfere with the function of the autoinjector 30.

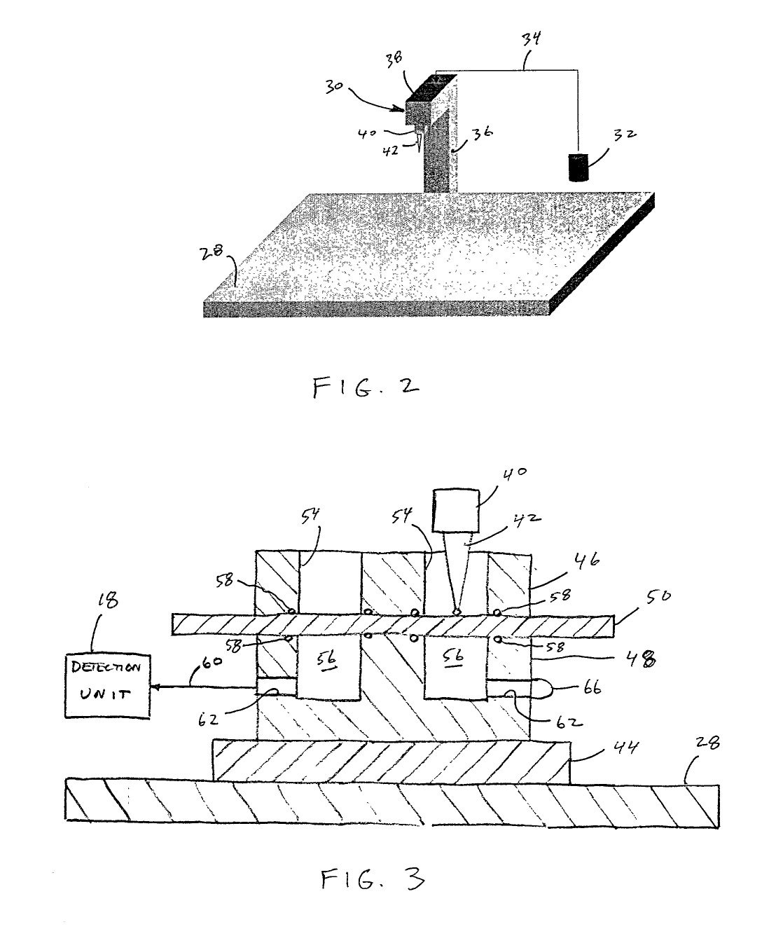

The upper swatch array block 46 is provided with an array of holes 54, and the lower swatch array block 48 is provided with a matching array of holes 56. As seen in FIG. 3, the upper block holes 54 extend completely through the upper block 46, while the lower block holes 56 extend from the upper surface of the lower block 48 but do not extend completely through. Each upper block hole 54 is aligned with a corresponding one of the lower block holes 56 when the upper block 46 is properly positioned on the lower block 48. An O-ring 58 is disposed around each one of the upper and lower block holes 54, 56 for sealing the holes at the respective interfaces with the material sample 50. The clamping mechanisms 52 compress the O-rings 58 to ensure sealing. Each corresponding pair of holes 54, 56 thus defines a separate test chamber for a different portion of the material sample 50. This arrangement in effect divides the material sample 50 into a plurality of distinct swatches for testing. Although six test chambers are shown in FIG. 1, it should be noted that the present invention is not limited to this number.

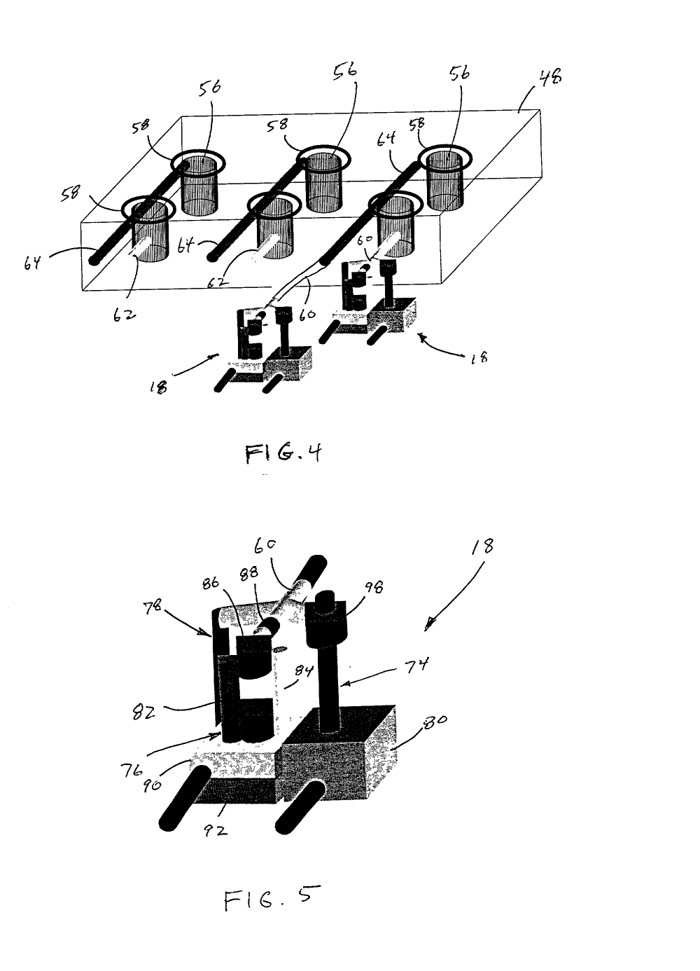

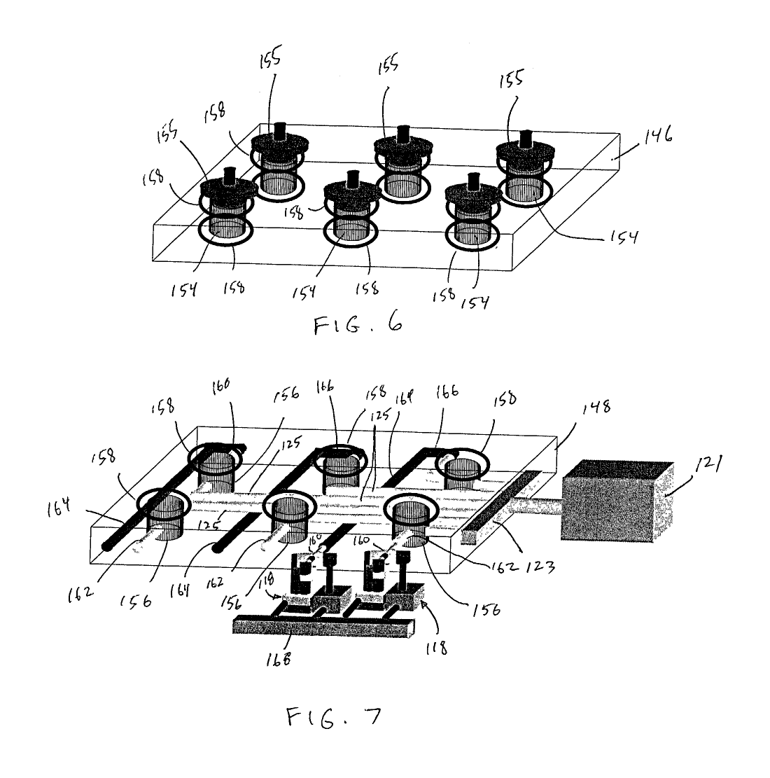

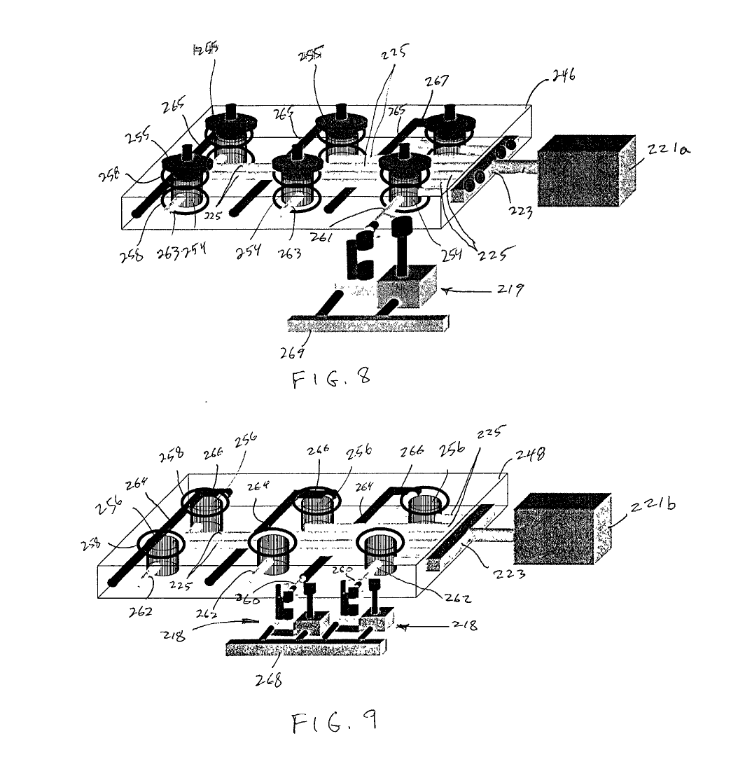

Each test chamber is associated with a respective one of the detection units 18, although only three detection units 18 are shown in FIG. 1 for clarity of illustration. The test chambers are in fluid communication with their corresponding detection unit 18 by means of a suitable conduit 60. Specifically, each lower block hole 56 has an exit passage 62 extending from the lower block hole 56 to the exterior of the lower block 48. In the case of the front row of lower block holes 56, the conduit 60 is connected directly between the outlet of the exit passage 62 and the detection unit 18. In the case of the back row of lower block holes 56, the outlet of the exit passage 62 is connected to a corresponding secondary passage 64 extending entirely through the lower block 48 (FIG. 4) by a “U” turn tube 66. The conduit 60 is then connected between the outlet of the secondary passage 64 and the detection unit 18. This arrangement locates all of the detection units 18 on the same side of the enclosure 12, which facilitates working on the detection units via box style gloves. Alternatively, the detection units for the front and back rows of test chambers could be located on opposite sides of the sample holding assembly, thereby eliminating the need for the secondary passages 64.

To dispense analyte onto the portion of the material sample 50 within a test chamber, a process also referred to as “spiking,” the syringe holder 40 is positioned over the selected test chamber through the robotic X, Y, Z positional control under the control of the controller 22. The syringe 42 is then lowered (through positional control of the support arm 38) into the upper block hole 54 as shown in FIG. 3. At the same time, the syringe 42 is actuated to dispense a precisely controlled amount of analyte, which forms as a drop on the tip of the syringe 42. The syringe 42 is lowered until the drop touches the surface of the material sample 50 inside the hole 54, which causes the drop to be deposited onto the upper surface of the material sample 50. (The material sample 50 is loaded into the sample holding assembly 16 such that the upper surface corresponds to the external surface during actual use, i.e., the surface facing away from skin.) The syringe 42 is then retracted. This sequence can be repeated multiple times until the desired amount of analyte is deposited. For example, the spiking process could be repeated ten times to deposit ten 1 microliter drops on the material sample 50 within the test chamber. Once the desired amount of analyte is deposited in one test chamber, the syringe holder 40 is positioned over the next test chamber for spiking that test chamber in the same manner. As mentioned above, the syringe holder 40 alternatively could be provided with an array of syringes instead of a single syringe. For example, the syringe holder 40 could have ten syringes nestled together to fit into the test chamber when lowered and thereby deposit ten drops of analyte onto the material sample 50 in one pass. Multiple syringes could also be arranged in a dispersed pattern matching the pattern of upper block hole to permit spiking of each test chamber simultaneously.

As mentioned above, each test chamber is associated with a respective one of the detection units 18. The inlet of each detection unit 18 is in fluid communication with the lower block hole 56 of the associated test chamber in the manner described above. All of the detection units 18, which are substantially identical, also include an outlet connected to a common exhaust manifold 68 that directs exhaust gases and vapors out of the enclosure 12 via a vacuum pump 70 connected to the exhaust manifold 68. A filter bank 72 is also provided for filtering the exhaust flow. The filter bank 72 includes a drying filter for drying the exhaust flow, a particulate filter unit, and one or more charcoal filters for removing analyte from the exhaust flow. Such filter systems are well known in the art.

A detection unit 18 is shown in more detail in FIG. 5. The detection unit 18 includes a first sensing branch 74, a second sensing branch 76 and a bypass branch 78, all connected in parallel between the conduit 60 and the exhaust manifold 68. The first sensing branch 74 includes a sensor cell 80 capable of sensing analyte. Gas from the test chamber is drawn through the first sensing branch 74 and the sensor cell 80 and then to the exhaust manifold 68 by the vacuum pump 70. The sensor cell 80 preferably comprises an array of sensors that produce distinct electrical signal outputs in response to the presence of the analyte. The sensor array can include semiconducting metal oxide (SMO) sensors, surface acoustic wave (SAW) sensors, chemical field effect transistors, surface plasmon resonance arrays, conducting polymer arrays, chemical hybrid sensor arrays, or any combination thereof. One suitable sensor cell is described in U.S. patent application Ser. No. 09/560,578, filed Apr. 27, 2000 and entitled “Materials Breakthrough Monitoring Sensor System.” This Application is assigned to the same assignee as the present application and is incorporated herein by reference.

The second sensing branch 76 includes an impinger 82 and an impinger bypass 84. A Y-valve 86 is provided for fluidly connecting either the impinger 82 or the impinger bypass 84 to the conduit 60. A check valve 88 is provided upstream of the Y-valve 86 to prevent backflow. A mass flow controller 90 is provided, and a pressure transducer 92 is included for detecting the pressure delta across the material sample 50 in the test chamber. Thus, gas from the test chamber can be directed through the impinger 82 and then to the exhaust manifold 68. The impinger 82 collects analyte in the gas in a conventional manner. The Y-valve 86 can be actuated to selectively direct the gas flow through the impinger bypass 84 to permit replacement of the impinger 82.

Using the two sensing branches in conjunction accommodates various sensing options and provides cross verification of the two detection results. Although the second branch 76 is described as using an impinger, it could alternatively other detection techniques such as sorbent tubes or gas chromatography. The bypass branch 78 provides an overflow function. For example, if the amount of flow through the conduit 60 for a desired delta pressure exceeds the combined desired flows for the first and second sensing branches 74, 76, then the extra flow can be directed to the exhaust manifold through the bypass branch 78.

The system 10 also includes in-situ calibration of the sensor cells 80. As shown in FIG. 1, a calibration gas source 94 supplies a calibration gas to the sensor cells 80 via a calibration gas manifold 96. Each sensor cell 80 has a Y-valve 98 that fluidly connects the sensor cell to either the corresponding conduit 60 for sensing operation or the calibration gas manifold 96 for calibration. The calibration gas has a known concentration of the analyte to enable calibration of the sensor cell 80.

The output signals from the sensor cells 80 are fed to the controller 22 for synchronized data collection, integration and processing. The controller 22 provides real time data and databasing capabilities. The controller, which can be a single PC, controls, operates and processes system parameters and allows for remote operation as well as communication capabilities. In particular, the controller 22 controls the input manifold 20, the autoinjector 30, the vacuum pump 70, flow rates, sensors and the various valves.

The system 10 as described above provides convective penetration testing. That is, a pressure differential is developed across the material being tested to evaluate resistance to convective (through-flow) penetration of analyte. The present invention also encompasses systems that evaluate resistance to diffusive (non-through-flow) penetration of analyte. These alternate systems are substantially similar to the system 10 described above, but have modified sample holding assemblies that handle the flow of conditioned lab air relative to the sample in a different manner. FIGS. 6 and 7 show the upper and lower swatch array blocks 146 and 148 of one alternative embodiment. The upper swatch array block 146 is provided with an array of holes 154, and the lower swatch array block 148 is provided with a matching array of holes 156. The upper block holes 154 extend completely through the upper block 146, while the lower block holes 156 extend from the upper surface of the lower block 148 but do not extend completely through. Each upper block hole 154 is aligned with a corresponding one of the lower block holes 156 when the upper block 146 is properly positioned on the lower block 148. An O-ring 158 is disposed around each one of the upper and lower block holes 154, 156 for sealing the holes at the respective interfaces with the material sample. The O-rings 58 are compressed by clamping mechanisms (not shown) to ensure sealing. Each corresponding pair of holes 154, 156 thus defines a separate test chamber for a different portion of the material sample. This arrangement in effect divides the material sample into a plurality of distinct swatches for testing. Although six test chambers are shown in FIGS. 6 and 7, it should be noted that the present invention is not limited to this number.

The test chambers are in fluid communication with their corresponding detection unit 118 by means of a suitable conduit 160. Specifically, each lower block hole 156 has an exit passage 162 extending from the lower block hole 156 to the exterior of the lower block 148. In the case of the front row of lower block holes 156, the conduit 160 is connected directly between the outlet of the exit passage 162 and the detection unit 118. In the case of the back row of lower block holes 156, the outlet of the exit passage 162 is connected to a corresponding secondary passage 164 extending entirely through the lower block 148 (FIG. 7) by a “U” turn tube 166. The conduit 160 is then connected between the outlet of the secondary passage 164 and the detection unit 118. This arrangement locates all of the detection units 118 on the same side of the enclosure, which facilitates working on the detection units via box style gloves. Alternatively, the detection units for the front and back rows of test chambers could be located on opposite sides of the sample holding assembly, thereby eliminating the need for the secondary passages 164.

The spiking process is substantially the same as that described above in connection with the first embodiment. That is, the system's autoinjector is actuated under the control of the controller to deposit a precisely controlled amount of analyte onto the surface of the material sample inside each upper block hole 154. After the analyte has been placed in the test chambers, a delta pressure connection cap 155 is mounted on each upper block hole 154 as shown in FIG. 6. The connection caps 155 block off the holes 154 to create a static environment on top of the material sample. Additional 0-rings 158 are provided to ensure sealing.

A flow through each test chamber is provided by a vapor generation system 121, which is very similar to the input manifold described above. That is, the vapor generation system 121 provides clean, conditioned air at a constant temperature and humidity. This is fed to the lower block hole 156 of each test chamber via a manifold 123 and a series of supply passages 125 running through the lower block 148. With this arrangement, there is no pressure delta across the material sample. As analyte begins to permeate the material samples in each test chamber, the flow carries it to the detection units 118, where the presence of analyte is detected in substantially the same manner described above, and then to the exhaust manifold 168.

FIGS. 8 and 9 show the upper and lower swatch array blocks 246 and 248 of another embodiment that evaluates resistance to diffusive penetration of analyte. The upper swatch array block 246 is provided with an array of holes 254, and the lower swatch array block 248 is provided with a matching array of holes 256. The upper block holes 254 extend completely through the upper block 246, while the lower block holes 256 extend from the upper surface of the lower block 248 but do not extend completely through. Each upper block hole 254 is aligned with a corresponding one of the lower block holes 256 when the upper block 246 is properly positioned on the lower block 248. An O-ring 258 is disposed around each one of the upper and lower block holes 254, 256 for sealing the holes at the respective interfaces with the material sample. The O-rings 58 are compressed by clamping mechanisms (not shown) to ensure sealing. Each corresponding pair of holes 254, 256 thus defines a separate test chamber for a different portion of the material sample. This arrangement in effect divides the material sample into a plurality of distinct swatches for testing. Although six test chambers are shown in FIGS. 8 and 9, it should be noted that the present invention is not limited to this number.

In this embodiment, each test chamber has two detection units 218, 219 associated with it. Specifically, a first detection unit 218 (FIG. 9) is in fluid communication with a corresponding lower block hole 256 by means of a suitable conduit 260, and a second detection unit 219 (FIG. 8) is in fluid communication with a corresponding upper block hole 254 by means of a suitable conduit 261. Each lower block hole 256 has an exit passage 262 extending from the lower block hole 256 to the exterior of the lower block 248. In the case of the front row of lower block holes 256, the conduit 260 is connected directly between the outlet of the exit passage 262 and the detection unit 218. In the case of the back row of lower block holes 256, the outlet of the exit passage 262 is connected to a corresponding secondary passage 264 extending entirely through the lower block 248 (FIG. 9) by a “U” turn tube 266. The conduit 260 is then connected between the outlet of the secondary passage 264 and the detection unit 218. Similarly, each upper block hole 254 has an exit passage 263 extending from the upper block hole 254 to the exterior of the upper block 246. In the case of the front row of upper block holes 254, the conduit 261 is connected directly between the outlet of the exit passage 263 and the detection unit 219. In the case of the back row of upper block holes 254, the outlet of the exit passage 263 is connected to a corresponding secondary passage 265 extending entirely through the lower block 248 (FIG. 8) by a “U” turn tube 267. The conduit 261 is then connected between the outlet of the secondary passage 265 and the detection unit 219.

This arrangement locates all of the detection units 218, 219 on the same side of the enclosure, which facilitates working on the detection units via box style gloves. Alternatively, the detection units for the front and back rows of test chambers could be located on opposite sides of the sample holding assembly, thereby eliminating the need for the secondary passages 264, 265.

A delta pressure connection cap 255 is mounted on each upper block hole 254 as shown in FIG. 8. The connection caps 255 block off the holes 254 to create a static environment on top of the material sample. Additional O-rings 258 are provided to ensure sealing. A first vapor generation system 221a is provided for the upper block 246. The first vapor generation system 221a provides a flow of conditioned air mixed with a controlled amount of vaporous analyte at a constant temperature and humidity. This mixture is fed to the upper block hole 254 of each test chamber via a manifold 223 and a series of supply passages 225 running through the upper block 246, thereby spiking the test chambers with analyte. Thus, the spiking process differs from that of the prior embodiments in that the analyte is not introduced with the system's autoinjector.

A second vapor generation system 221b is provided for the lower block 248. The second vapor generation system 221b provides clean, conditioned air at a constant temperature and humidity. This air is fed to the lower block hole 256 of each test chamber via a manifold 223 and a series of supply passages 225 running through the lower block 248. With this arrangement, there is no pressure delta across the material sample. As analyte begins to permeate the material samples in each test chamber, the flow through the lower block holes 256 carries it to the detection units 218, where the presence of analyte is detected in substantially the same manner described above, and then to a first exhaust manifold 268. The flow through the upper block holes 254 carries analyte to the detection units 219, where the presence of analyte is detected in substantially the same manner described above, and then to a second exhaust manifold 269.

While specific embodiments of the present invention have been described, it will be apparent to those skilled in the art that various modifications thereto can be made without departing from the spirit and scope of the invention as defined in the appended claims.

What is claimed is:

1. A system for testing chemically resistant materials, said system comprising:
a sample holding assembly for holding a sample of a material to be tested, said sample holding assembly defining a plurality of test chambers wherein each test chamber has a portion of said sample disposed therein;
means for dispensing a controlled amount of an analyte into each test chamber; and
a plurality of analyte detection units, each one of said detection units being in fluid communication with a corresponding one of said test chambers.

2. The system of claim 1 further comprising a gas tight enclosure, said sample holding assembly, said means for dispensing and said analyte detection units being located inside said enclosure.

3. The system of claim 2 further comprising means for introducing conditioned air into said enclosure.

4. The system of claim 1 wherein said means for dispensing includes an robotic autoinjector having an at least one analyte dispensing syringe mounted for motion along three mutually orthogonal axes.

5. The system of claim 4 further comprising a controller for controlling positioning of said syringe.

6. The system of claim 1 wherein said sample holding assembly includes a lower swatch array block and an upper swatch array block stacked on top of said lower swatch array block with said sample sandwiched therebetween, said lower swatch array block having a first plurality of holes formed therein and said upper swatch array block having a second plurality of holes formed therein, wherein each one of said first holes is aligned with a corresponding one of said second holes to define one of said test chambers.

7. The system of claim 6 further comprising means for clamping said upper swatch array block to said lower swatch array block.

8. The system of claim 6 wherein said means for dispensing includes an robotic autoinjector comprising:
a column movable along a first axis;
a support arm mounted to said column and movable along a second axis that is orthogonal to said first axis, said support arm extending over said sample holding assembly;
a syringe holder mounted to said support arm and movable along a third axis that is orthogonal to both said first and second axes; and
at least one analyte dispensing syringe mounted to said syringe holder.

9. The system of claim 6 wherein each one of said detection units being in fluid communication with a corresponding one of said first plurality of holes.

10. The system of claim 6 wherein said plurality of detection units is divided into first and second groups, each one of said detection units of said first group being in fluid communication with a corresponding one of said first plurality of holes and each one of said detection units of said second group being in fluid communication with a corresponding one of said second plurality of holes.

11. The system of claim 1 wherein each one of said detection units includes a sensor cell having an array of sensors that produce distinct electrical signal outputs in response to the presence of said analyte.

12. The system of claim 1 wherein each one of said detection units includes a first sensing branch and a second sensing branch that operate in parallel, said first sensing branch including a first means for sensing analyte and said second sensing branch including a second means for sensing analyte.

13. The system of claim 12 wherein said first means for sensing analyte includes a sensor cell having an array of sensors that produce distinct electrical signal outputs in response to the presence of said analyte.

14. The system of claim 13 wherein said second means for sensing analyte includes an impinger.
